(19) United States
(12) Patent Application Publication
(10) Pub. No.: US 2013/0052689 A1
(43) Pub. Date: Feb. 28, 2013

(54) Methods and Systems for Producing Products Using Engineered Ammonia Oxidizing Bacteria

(71) Applicants: Scott Banta, New York, NY (US); Kartik Chandran, New York, NY (US); Alan West, Tenafly, NJ (US)

(72) Inventors: Scott Banta, New York, NY (US); Kartik Chandran, New York, NY (US); Alan West, Tenafly, NJ (US)

(21) Appl. No.: 13/650,824

(22) Filed: Oct. 12, 2012

Related U.S. Application Data
(63) Continuation-in-part of application No. PCT/US2011/032317, filed on Apr. 13, 2011.
(60) Provisional application No. 61/561,547, filed on Nov. 18, 2011.
(60) Provisional application No. 61/323,595, filed on Apr. 13, 2010.
(60) Provisional application No. 61/426,020, filed on Dec. 22, 2010.

(51) Int. Cl. C12P 7/16; C12P 19/00; C12P 13/04; C12P 7/04; C12M 1/42

(52) U.S. Cl. 435/72; 435/157; 435/160; 435/289.1; 435/106

(57) ABSTRACT

Methods and systems for producing a biofuel using genetically modified ammonia-oxidizing bacteria (AOB) are disclosed. In some embodiments, the methods include the following: providing an AOB that have been genetically modified to include a particular metabolic pathway to enable them to generate a particular biofuel or chemical; feeding a first source of ammonia to the AOB; feeding carbon dioxide to the AOB; and producing at least the biofuel or chemical, nitrite, and an AOB biomass. In some embodiments, the methods and systems include the following: a bioreactor including AOB that have been genetically modified to include a particular metabolic pathway to enable them to generate a particular biofuel; a first source of ammonia; a source of carbon dioxide; and a electrochemical reactor that is configured to electrochemically reduce nitrite produced in the bioreactor to a second source of ammonia.

### STATEMENT REGARDING FEDERALLY SPONSORED RESEARCH OR DEVELOPMENT

This invention was made with government support under DE-AR0000060 awarded by Advanced Research Projects Agency of the Department of Energy. The government has certain rights in the invention.

### BACKGROUND

There has been interest in the development of liquid biofuels as these processes have the potential to directly fix carbon dioxide into transportation fuels, which is potentially carbon neutral and politically attractive. Cellulose based biofuels including bioethanol, algae-derived lipids, cyanobacteria, and algae derived hydrogen (H₂) are among the most studied biofuels. Despite the promise of these technologies and processes, there are specific limitations that preclude their wide-spread application. For example, post-processing of algal cells and derived lipids imposes higher production costs on algal biodiesel. The production rates of H₂ from cyanobacteria still remains low and productivity needs to be improved. Genetically engineered photosynthetic organisms have also been explored for bioethanol production. However separation of ethanol from the aqueous phase remains a challenge.

Microbial fuel cells have been under investigation and development for more than a century, as the use of cells to harvest electrical energy from waste streams is attractive for many reasons. In a biofuel cell, biological catalysts are used on an anode to oxidize biofuels, and a cathode is created that can use the generated electrons to reduce oxygen to water. These systems can either be microbial with living cells on the electrodes, or they can be enzymatic systems, with purified enzymes on the electrodes. In both designs, power can be generated from the oxidation of biofuels, and there are many advantages to these systems over conventional fuel cells and other power generation schemes. However, much research still needs to be done with microbial fuel cells to make them practical and cost-efficient. A significant limitation for both enzymatic and microbial fuel cells is the need for mediators to enable electrical contact between the biological components and inorganic electrode. In some microbial systems, these mediators are made by the organisms themselves, and in other technologies, synthetic mediators are added to the system. In some systems, cells must make physical contact with the electrodes for electron transfer. This can be a significant limitation as it reduces the cellular mass that can be used for biochemical conversion.

### SUMMARY

Living cells have the ability to reproduce and maintain their catalytic machinery, and their metabolic pathways can be rationally altered to meet desired process objectives. But, efficient electron transfer from the electrode to the organism can limit metabolic production, and the use of mediating species can result in a process that is not economically viable. One way to address these limitations is to explore alternative organisms that naturally utilize mediators that are more attractive. The disclosed subject matter includes the metabolic engineering of chemolithoautotrophic ammonia-oxidizing-bacteria (AOB), such as Nitrosomonas europaea, to develop a process that can overcome these limitations. AOB have the natural ability to fix carbon dioxide while oxidizing ammonia to nitrite.

Referring to FIGS. 1 and 2, aspects of the disclosed subject matter include the use of engineered strains of the AOB, e.g., N. europaea 19718, for the production of biofuels. AOB fix carbon dioxide for cell-synthesis while deriving energy from the oxidation of ammonia to nitrite. The nitrite produced upon ammonia oxidation can be electrochemically reduced back to ammonia in an electrochemical reactor, and additional ammonia can be added from any ammonia-rich stream, e.g., one derived from a wastewater treatment process. In this way, the AOB can be grown efficiently in a bioreactor using ammonia as the mediator.

Other aspects of the disclosed subject matter include a downstream two-stage bioreactor system for the fermentation of the produced bacterial biomass directly to additional useful biofuels in order to improve the viability of the process.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a schematic diagram of methods and systems according to some embodiments of the disclosed subject matter;

FIG. 2 is a schematic diagram of methods and systems according to some embodiments of the disclosed subject matter;

FIG. 3 is a schematic diagram of systems according to some embodiments of the disclosed subject matter;

FIG. 4 is a chart of a method according to some embodiments of the disclosed subject matter;

FIG. 5 is a schematic diagram of methods and systems according to some embodiments of the disclosed subject matter;

FIG. 6 is a diagram showing production of isobutanol via an AOB having a modified genetic sequence according to some embodiments of the disclosed subject matter;

FIG. 7 is a chart of a method according to some embodiments of the disclosed subject matter;

FIG. 8 is a chart showing current efficiency of nitrite reduction to ammonia as a function of percentage of nitrite converted to ammonia in spent media from the bioreactor, the inset shows the current efficiencies for 20 mM sodium nitrite in 100 mM phosphate buffer as a function of applied potential in batch experiments in an undivided three electrode cell with a glassy carbon cathode;

FIG. 9 is a chart showing the change in catholyte pH for different anolytes: 50 mM phosphate buffer, pH 7.0+50 mM sodium nitrite (filled circles), 0.5 M sulfuric acid (empty circles), 2.0 M sulfuric Acid (triangles) and 3 M sulfuric acid (rectangles);

FIG. 10 is a table showing the half cell and overall reactions assuming oxygen evolution at the anode at different anolyte pH;

FIG. 11 is a chart showing potential vs. time for constant current experiments in 100 mM phosphate buffer pH 7.0+1 M; sodium nitrite at nickel RDE at 1 mA/cm², 5 mA/cm₂ (dotted line) and 10 mA/cm²;

FIG. 12 is a chart showing ammonia removal efficiency and observed biomass yield for continuous flow cultivated Nitrosomonas europaea, Period 1 was defined by growth on synthetic media prepared from ACS grade chemicals, Period 2 was defined by cultivation on spent media from Period 1 that was electrochemically regenerated, and Period 3 was defined by cultivation on spent media from Period 2 that was electrochemically regenerated;

FIG. 13 is a chart showing current as a function of sweep rate for nitrite reduction on nickel (sweep rate was 5 my s-1) in 100 mM phosphate buffer pH 7.0+1 M sodium nitrite and 100 mM phosphate buffer pH 7.0+1 M sodium nitrite+50 mg/L isobutanol at different rotation speeds;

FIG. 14 is an enlarged photograph of monoclonal Nitrosomonas europaea cells (strain pRNIT-1) expressing green fluorescent protein under the control of the RubisCO large subunit promoter (PrbcL);

FIG. 15 shows results from qualitative-RT-PCR confirming the expression of A) kdcA and B) adH2 in transformed Nitrosomonas europaea, Lanes 1 and 5—100 bp DNA ladder, Lane 2—kdcA gene transcript from N. europaea culture transformed with pRKivd-1, Lane 3—kdcA gene transcript from N. europaea culture transformed with pRKA-1, Lane 4—kdcA positive control (amplification from plasmid DNA (purified pRKivd-1)), Lane 6—adh2 gene transcript from N. europaea culture transformed with pRADH2-1, Lane 7—adh2 gene transcript from N. europaea culture transformed with pRKA-1, and Lane 8—adh2 positive control (amplification from plasmid DNA (purified pRADH2-1));

FIG. 16 is a chart of concentration vs. time for Isobutyraldehyde for batch cultured N. europaea cells transformed with pRKivd-2; and

FIG. 17 is a chart of ammonia removal efficiency and isobutyraldehyde production from flow cultivated Nitrosomonas europaea cells transformed with pRKivd-1.

### DETAILED DESCRIPTION

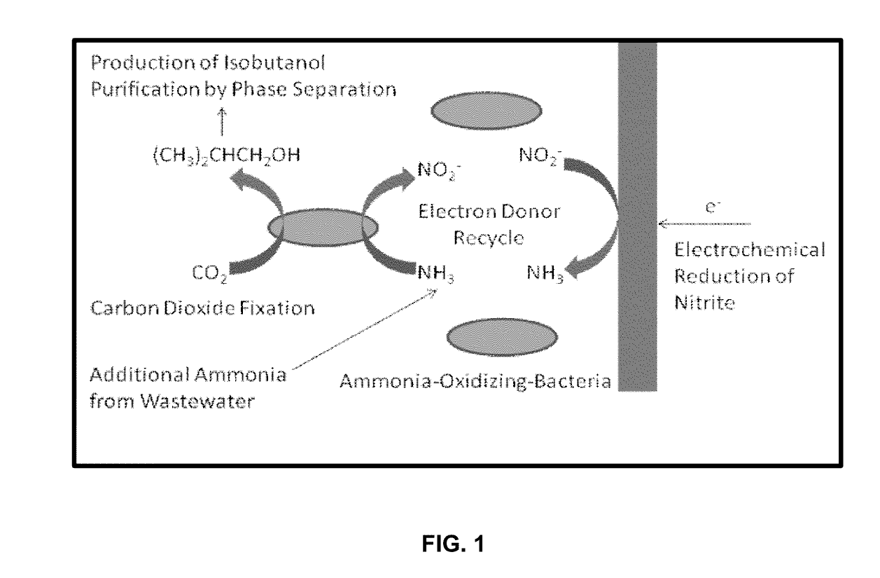

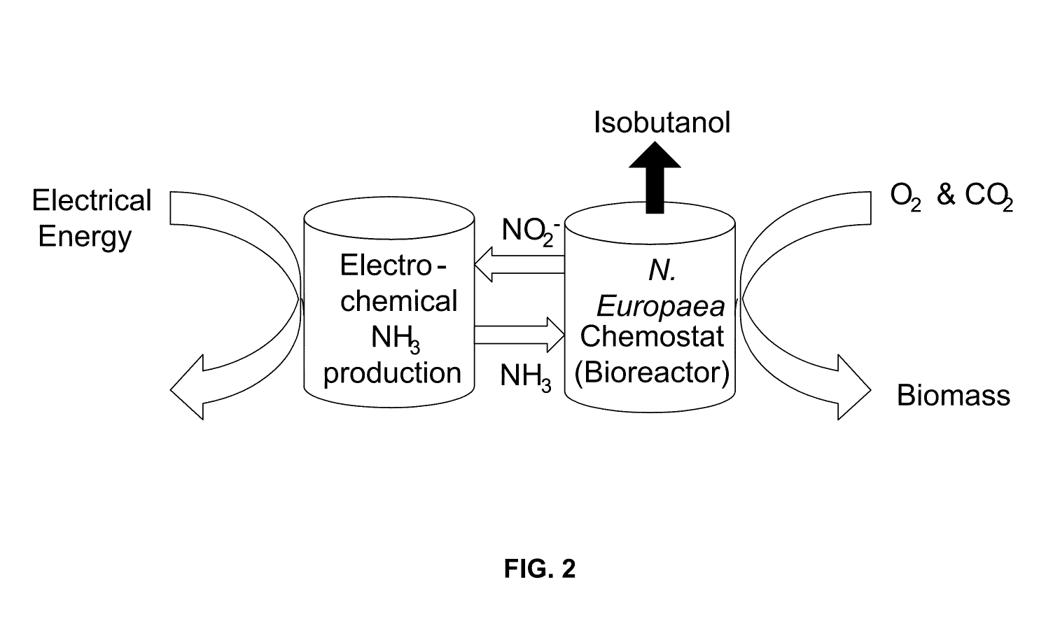

Referring again to FIGS. 1 and 2, aspects of the disclosed subject matter include methods and systems that include the application of chemolithoautotrophic AOB for concomitant carbon dioxide fixation, conversion of the carbon dioxide to a biofuel such as isobutanol, and oxidation of ammonia to nitrite. The nitrite produced upon ammonia oxidation is electrochemically reduced back to ammonia. Additional ammonia can be added from other ammonia-rich sources, e.g., derived from a wastewater treatment process. Other sources of ammonia will typically be sterilized to ensure it does not foul the AOB purity and bioreactor environment. Metabolic engineering is used to introduce a new pathway into the bacteria that starts with the precursors for amino acid synthesis to create butanols, e.g., isobutanol, etc.

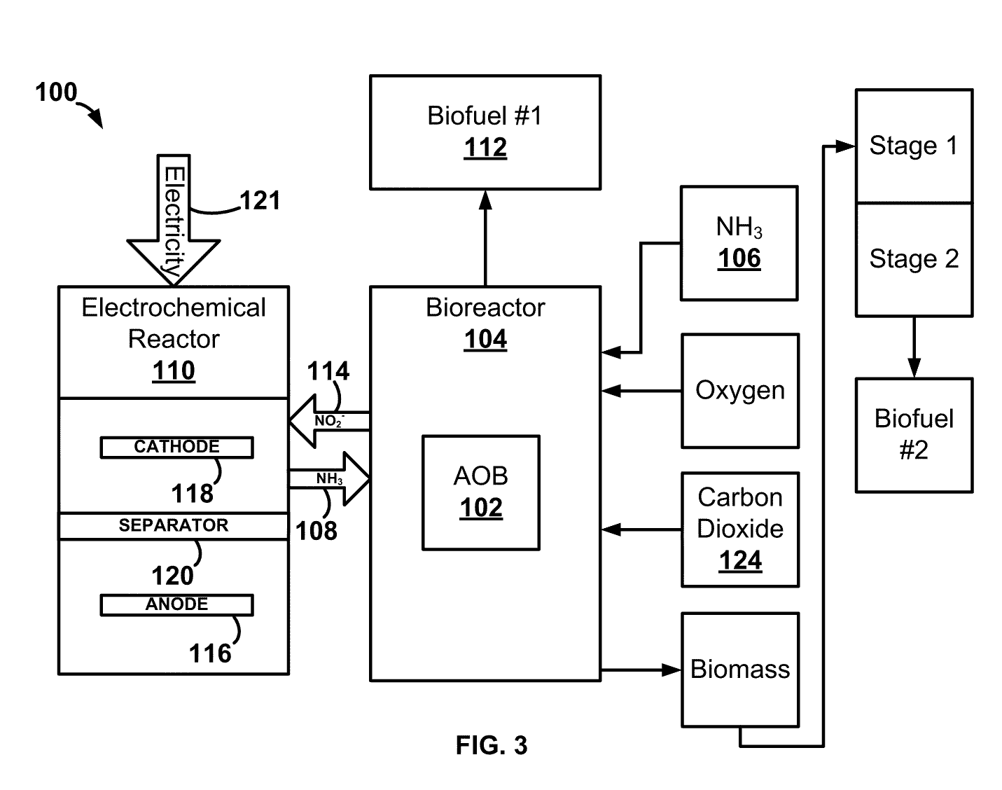

Referring now to FIG. 3-7, some embodiments include systems and methods for producing products such as biofuels and chemicals. As shown in FIG. 3, some embodiments include a system 100 for producing biofuels using genetically modified AOB 102 grown in a bioreactor 104 that are fed ammonia and carbon dioxide. The ammonia provides electrons to the AOB and the carbon dioxide is used as a base material to be fixed into a biofuel or chemical. Initially, the ammonia is typically provided from a first source 106 that is external to system 100, e.g., an ammonia-rich stream derived from a wastewater treatment process, etc., but in fluid communication with bioreactor 104. Typically, but not always, the ammonia used in system 100 is substantially provided by a second source 108 that is generated by an electrochemical reactor 110. Second source 108 of ammonia serves as a mediator for transferring electrons to AOB 102. In some embodiments, substantially all of the ammonia used by bioreactor 104 is provided by a source external to system 100.

Bioreactor 104 includes AOB 102 that have been genetically modified to include a particular metabolic pathway to enable them to generate a particular biofuel 112. The operating parameters of bioreactor 104 are typically optimized to maximize the production of nitrite 114 and minimize the production of nitrate. In some embodiments, bioreactor 104 will be configured so as to be fed 40 mM of ammonia, 10 mM of nitrate, 300 mM of phosphate, 80 g/L of carbonate, and trace metals (at micro-g/L levels). In some embodiments, the pH will likely be maintained in the range of about 7.5 to 8.0 and temperature at about 30 degrees Celsius. Methods and systems according to the disclosed subject matter have operating conditions that are optimized for optimal yield, conversion, etc. Bioreactors included in methods and systems according to the disclosed subject matter are typically operated in a continuous flow mode to maximize the conversion of the substrates to the products. Ammonia and nitrite toxicity, leaching of ions from the electrodes, and product (biofuel) toxicity are mitigated in the methods and systems according to the disclosed subject matter.

Nitrite 114, which is generated in bioreactor 104, is introduced to electrochemical reactor 110, which is in fluid communication with the bioreactor. Electrochemical reactor 110 includes electrodes, i.e., an anode 116 and a cathode 118, a separator 120, and source of electrical energy 121. In some embodiments, cathode 118 is formed substantially from nickel or glassy carbon and anode 116 is formed from materials known in the art. In some embodiments, flow through or flow by porous electrodes are used.

Electrochemical reactor 110 is typically configured to electrochemically reduce nitrite 114 to second source 108 of ammonia using source of electrical energy 121. In system 100, nitrite 114 will be continually regenerated back to ammonia, i.e., second source 108, and the recycle loop can be theoretically closed without the need for additional ammonia input from first source 106 beyond startup.

In some embodiments, a portion of the ammonia provided to bioreactor 104 is obtained from an ammonia-rich stream derived from a wastewater treatment process and a portion is obtained from electrochemical reactor 110.

Some embodiments of the disclosed subject matter include systems having holding tanks for the ammonia rich streams and nitrite rich streams to enable the electrochemical production of ammonia to operate independently of the bioreactor to take advantage of the transient pricing and availability of electricity. For example, at times during the day when electricity is least expensive, the electrochemical system would produce as much ammonia as possible to be stored and used slowly by the bioreactor, which will be operating continuously. This solves a major limitation encountered in photobioreactors where interruptions in light can negatively impact the process.

System 100 includes a source 124 of carbon dioxide that is in fluid communication with bioreactor 104. In some embodiments, source 124 is carbon dioxide removed from air or energy plant emissions. In some embodiments, either in place of or in addition to carbon dioxide, carbonate, e.g., from mineral sources, is fed to bioreactor 104.

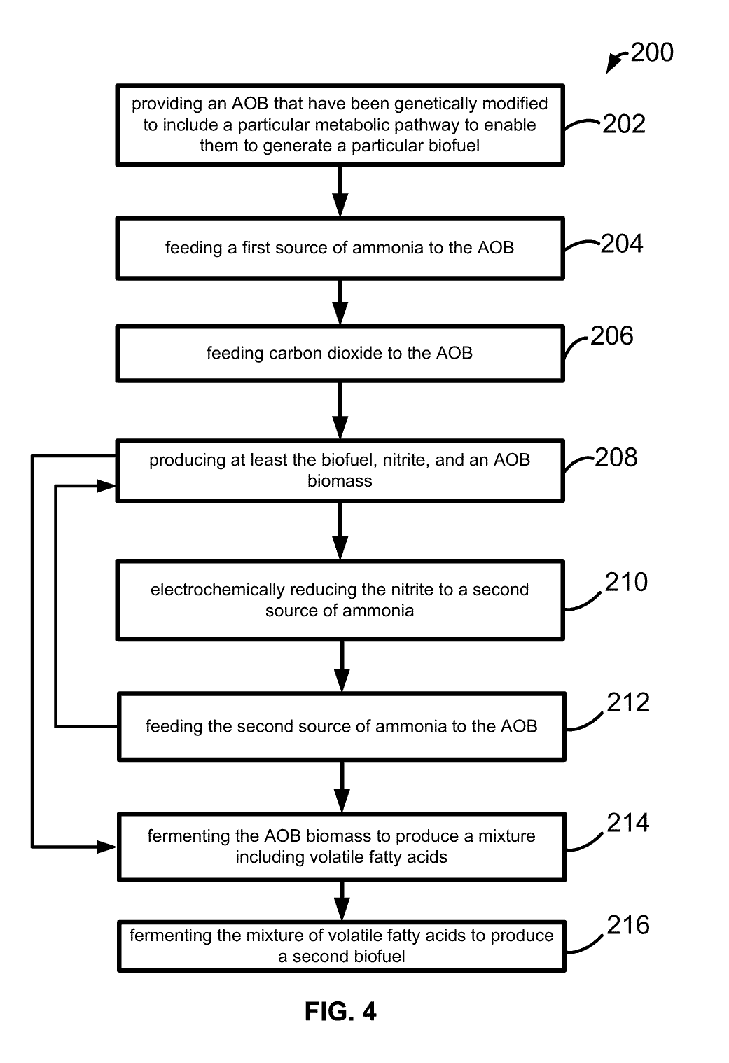

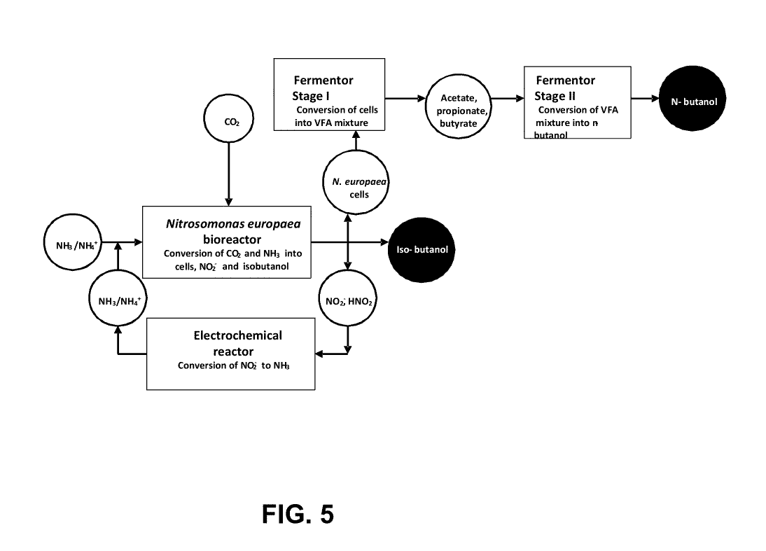

Referring now to FIGS. 4-7, some embodiments include a method 200 for producing a biofuel using genetically modified AOB. As shown in FIG. 4 (and schematically in FIG. 5), at 202, AOB that have been genetically modified to include a particular metabolic pathway to enable them to generate a particular biofuel are provided.

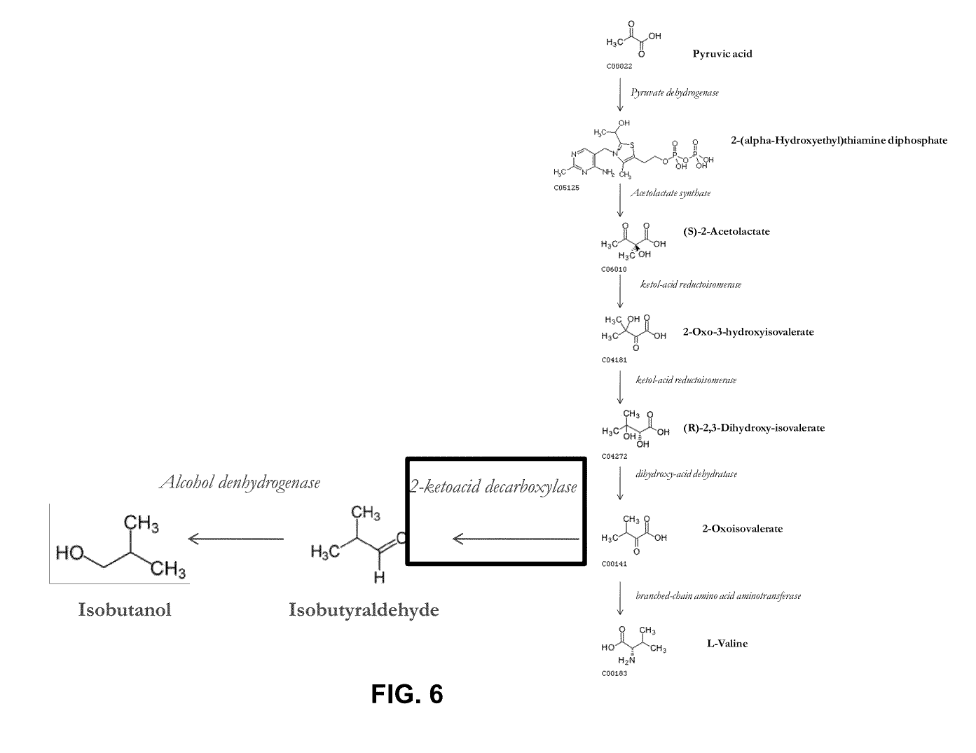

As shown best in FIG. 6, in some embodiments, the AOB is substantially Nitrosomonas europaea and the AOB are genetically modified by including at least one of a 2-keto-acid decarboxylase gene (outlined by box) and an alcohol dehydrogenase gene or similar. The production of isobutanol in prokaryotic hosts begins with the amino acid biosynthesis pathways. These pathways produce 2-keto acids, and these are converted to aldehydes using a 2-keto-acid decarboxylase. Alcohol dehydrogenase is then used to convert the aldehydes to alcohols. In the case of isobutanol, the valine biosynthesis pathway is used, and the starting precursor is 2-keto-isovalerate.

In some embodiments, the AOB provided are genetically modified to be able to utilize hydrogen as an electron donor. The use of hydrogen as a mediator improves system efficiency because hydrogen may be cogenerated with ammonia during the electrochemical regeneration step. There are various hydrogenase enzymes from different organisms that can be used in microbial biohydrogen production. But other hydrogenase enzymes, found in organisms such as Metallosphaera sedula and hodopseudomonas palustris, enable hydrogen uptake and its use as a reductant.

Referring again to FIGS. 4 and 5, at 204, a first source of ammonia is fed to the AOB. At 206, carbon dioxide is fed to the AOB. At 208, a biofuel, nitrite, and an AOB biomass are produced. In some embodiments, nitrite production is maximized and nitrate production is minimized during 208. In some embodiments, the biofuel is one of isobutanol, a long chain alcohol, or an alkane. At 210, the nitrite produced is electrochemically reduced to a second source of ammonia. Hydrogen is also often produced while electrochemically reducing the nitrite. Next, at 212, the second source of ammonia and the hydrogen are fed to the AOB. The second source of ammonia serves as a mediator for transferring electrons to the AOB. Then, the process returns to 208 where additional biofuel, nitrite, and AOB biomass are produced.

Some embodiments of the disclosed subject matter include methods and systems that do not include the electrochemical regeneration of ammonia. For example, where a feed rich in ammonia exists, the conversion of ammonia and CO₂ to a valuable product (biofuel or other chemical) can be achieved without electrochemical regeneration of ammonia. Typical waste streams suitable for this technology would have high ammonia concentrations (in the range of 200-7000 mg-N/L or higher). In some cases, e.g., when the chemical product being produced is very valuable, purchased ammonia in the form of gas or a salt such as ammonium carbonate will be used as a feedstock, thus eliminating the need for the electrochemical regeneration of ammonia.

At 214, the AOB biomass is fermented to produce a mixture including volatile fatty acids (VFA), e.g., including acetate, propionate, and butyrate. Then at 216, the mixture of VFAs is fermented to produce a second biofuel, e.g., butanol or similar. The overall fermentation process is split into two stages, i.e., 214 and 216, to allow independent optimization of VFA production and butanol production, which involve different microbial organisms and pathways. In some embodiments, the fermentation of cellular biomass to a mixture of VFA in stage 1 will be conducted at 37 degrees Celsius and is anticipated to be catalyzed by a broad variety of fermentative bacteria. In some embodiments, stage 2 will also be operated at 37 degrees Celsius, and will be catalyzed by an axenic culture of Clostridium beijerinckii BA101. Operation of stage 2 at 37 degrees Celsius has been shown to yield higher butanol synthesis rates compared to lower temperatures. Typically, the main operational parameter of engineered biological reactors is the solids retention time (SRT), which governs the physiological activity and metabolic rate of the resident microorganisms therein. In some embodiments, to achieve VFA synthesis from cell biomass, an SRT of about 3 to 4 days is used. However, conversion of VFA (including butyrate) to butanol requires a cell residence time in the range of about 0.4 to 1 day.

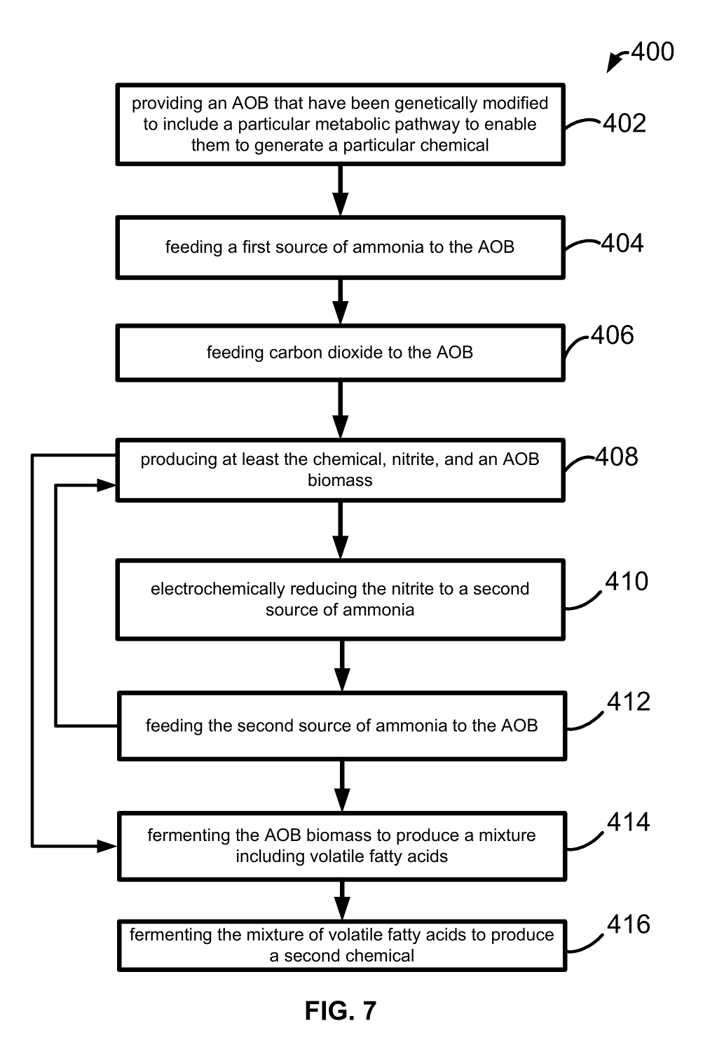

Referring now to FIG. 7, some embodiments include a method 400 for producing a chemical using genetically modified AOB. At 402, AOB that have been genetically modified to include a particular metabolic pathway to enable them to generate a particular chemical are provided. At 404, a first source of ammonia is fed to the AOB. At 406, carbon dioxide is fed to the AOB. At 408, a chemical, nitrite, and an AOB biomass are produced. At 410, the nitrite produced is electrochemically reduced to a second source of ammonia. Hydrogen is also often produced while electrochemically reducing the nitrite. Next, at 412, the second source of ammonia and the hydrogen are fed to the AOB. Then, the process returns to 408 where additional chemical, nitrite, and AOB biomass are produced. At 414, the AOB biomass is fermented to produce a mixture including volatile fatty acids, e.g., including acetate, propionate, and butyrate. Then at 416, the mixture of volatile fatty acids is fermented to produce a second chemical such as a valuable commodity chemical, specialty chemicals, feedstocks such as acids, amino acids, carbohydrates, and other molecules.

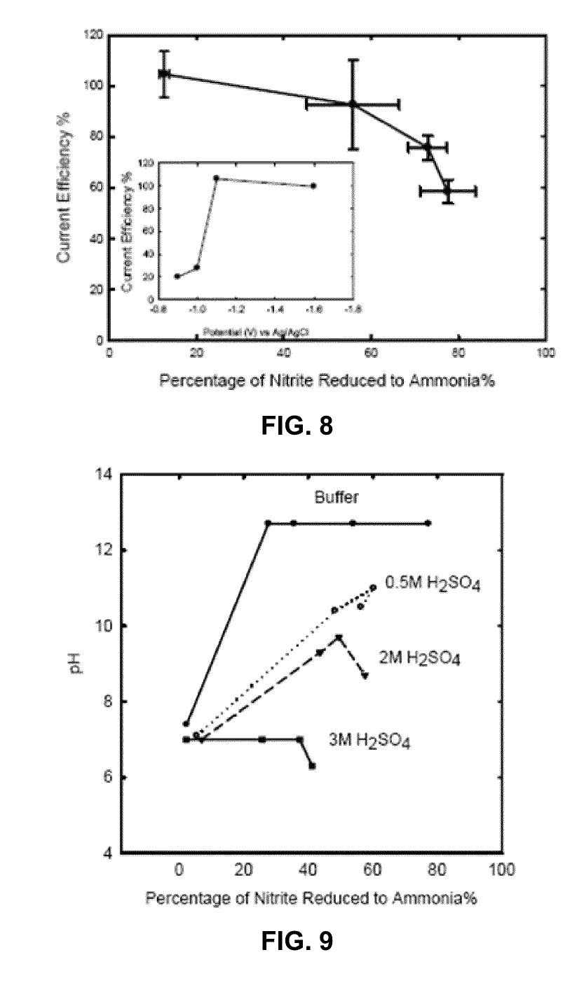

Referring now to FIGS. 8-12, testing of some embodiments of the disclosed subject matter was performed to demonstrate the growth of Nitrosomonas europaea fed with CO₂ in a bioreactor using the electrochemical reduction of nitrite to ammonia. Referring now to FIG. 8, for some embodiments, testing data shows the current efficiency of nitrite reduction to ammonia influent media from the N. europaea chemostat. The initial current efficiency is 104.5%±9.0. However, at 78% conversion of nitrite to ammonia, the current efficiency decreases to 58.4%±4.7. This decrease is assumed to be due to mass transfer limitations at high conversions of nitrite and can be avoided by further optimizing the operating conditions at higher conversions. The FIG. 8 inset shows the current efficiency measured in constant applied potential experiments with an undivided three electrode cell set-up for 20 mM of nitrite in a phosphate buffer. As shown, near 100% current efficiencies can be obtained under optimal operating conditions with lower nitrite concentrations.

Previously, one of the challenges of coupling reactors according to the disclosed subject matter was determined to be the increase in pH in the electrochemical reactor during reduction of nitrite to ammonia. As a short term solution to this problem, a pH controller was used to keep the catholyte pH constant by adding 5M hydrochloric acid to catholyte. This resulted in ˜3% dilution of the catholyte. Referring now to FIG. 9, where changes in catholyte pH for different anolyte solutions varying from phosphate buffer at pH 7.0 to 3M sulfuric acid are shown, the long term solution is to replace the cation exchange membrane with a Nafion membrane which is ˜3000 times more selective to H+ compared to other cations and increase the H+ concentration in the anolyte.

For long term operation, it is recognized that the system will require an acid source to maintain the proper pH. The precise amounts are not known because the bioreactor operation leads to a net production of protons (in a less predictable manner). As additional process data are available, acid consumption will be an important factor for establishing process feasibility. Preliminary studies may suggest however, that electrical power requirements for the electrochemical reaction may be the primary economic consideration. The power requirements can be calculated using Eq 1.1:

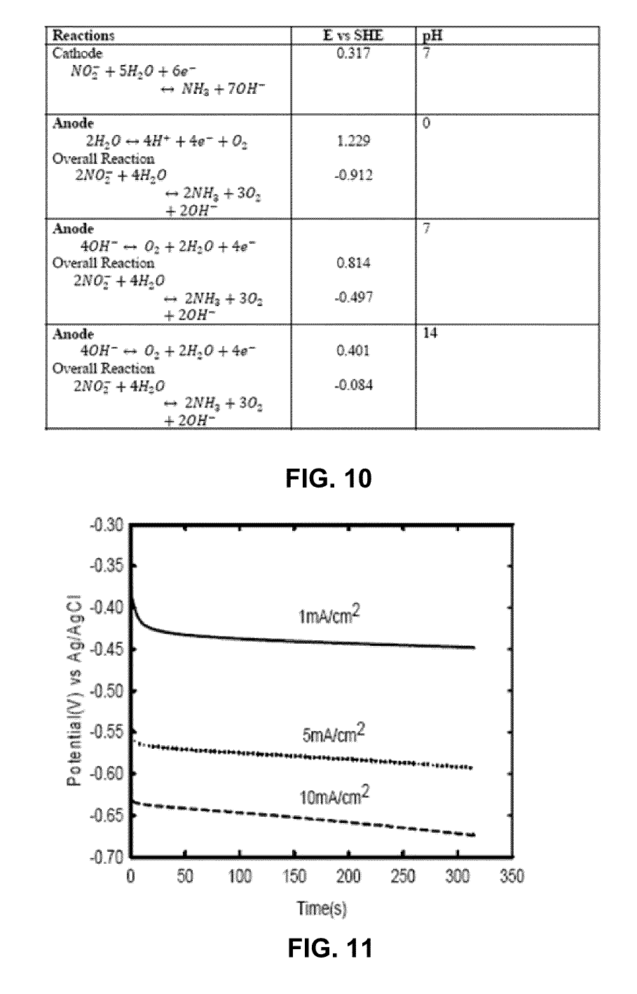

P═IV (Eq 1.1)

where P is power, I is the current required which can be calculated using Faraday's law and corrections based on current efficiency measurements, and V is the cell potential. FIG. 10 shows the half cell and overall reactions assuming oxygen evolution at the anode at different anolyte pH.

For a more accurate calculation of the power requirements, the over potentials were determined experimentally for copper, glassy carbon, and nickel electrodes. Among the cathodes materials tested, nickel exhibited the lowest over potential. FIG. 11 shows the potential of a nickel rotating disk electrode at different current densities. Over potential at the cathode can be calculated by subtracting the thermodynamic potential from the operating potential. For example, at an applied current density of 5 mA/cm², the operating potential is −0.56V vs. Ag/AgCl and the thermodynamic half-cell potential is 0.120 V vs. Ag/AgCl (0.317 vs. SHE). Therefore, the cathode over potential is (−0.56-0.120)=−0.68V. At the anode, oxygen evolution is taking place. This reaction has been optimized for various industrial processes such as water electrolysis in alkaline solutions and metal electro winning in acidic electrolytes. Over potentials as low as 0.400V in sulfuric acid solutions were reported with composite electrodes. Using these over potentials (680 mV for the cathode and 400 mV for the anode) and the thermodynamic half-cell potentials at the appropriate pH, one can estimate that a cell potential of around 2.0V is realizable in a practical system. However, as the overall system is developed, improvements in the cathode may lead to further reductions in potential.

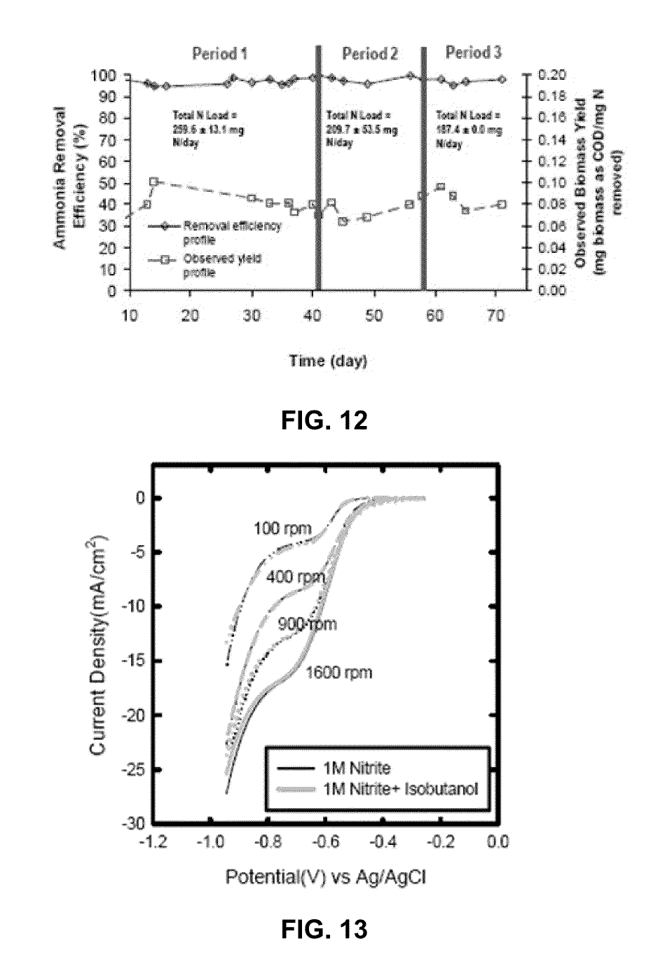

Referring now to FIG. 12, in test of some embodiments of the disclosed subject matter, electrochemically produced ammonia was successfully used to cultivate wild type N. europaea in continuous flow reactors. Ammonia removal efficiency was maintained above 96% during all periods of operation (Period 1=97.2±1.5%; Period 2=97.9±1.3% Period 3=95.5±2.7%). Comparison of ammonia removal efficiency for cells cultured with electrochemically reduced media (ElecM) versus cells cultured on synthetic media (SynM) indicated that there was a small decrease in efficiency as cultures were sequentially propagated on ElecM; however, this change was not statistically different at the 95% confidence interval (p>0.18 for all comparisons). Observed biomass yields during these experiments were also statistically similar regardless of the media utilized for growth (Period 1=0.08±0.02 mg biomass as COD/mg N; Period 2=0.09±0.02 mg biomass as COD/mg N; Period 3=0.08±0.01 mg biomass as COD/mg N; p>0.05 for all comparisons). Similarly, the calculated free energy efficiency (ratio of energy present as organic carbon divided by energy produced by ammonia oxidation to nitrite) during these experiments was steady and averaged 4.81±0.37% (p>0.05 for all comparisons).

Referring now to FIGS. 13-17, some embodiments of the disclosed subject matter were tested to characterize and optimize the electrochemical system when operating with the bioreactor containing genetically modified N. europaea cells to maximize isobutanol production. FIG. 13 shows the effect of the presence of isobutanol during nitrite reduction. As isobutanol is added to the nitrite solutions, no significant change is observed in the linear sweeps. The concentration of isobutanol used in this study is based on the half maximal inhibitory concentration of isobutanol for N. europaea. Previous experiments with antibiotics suggested that no cathodic activity was observed on gold or platinum electrodes in electrolytes ranging from 1M sulfuric acid to 0.1 M sodium carbonate. Preliminary linear-sweep experiments suggest that the antibiotics remain inert in media. These findings show that there should not be an impact on the performance of the electrochemical reactor when the chemostat is operated with transformed cells that require antibiotics and are producing isobutanol.

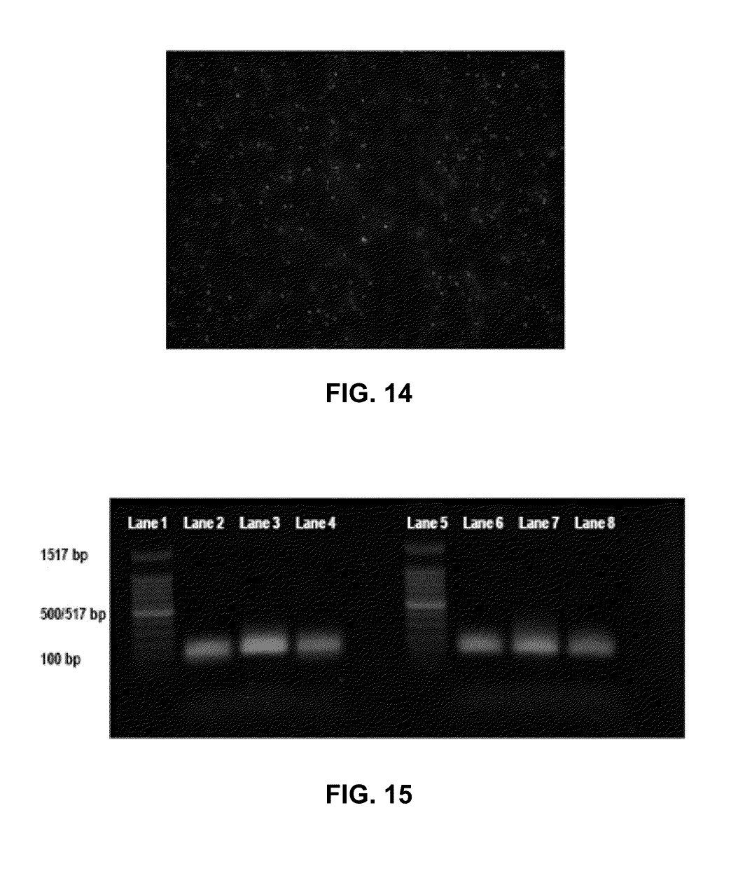

Referring now to FIG. 14, testing of embodiments according to the disclosed subject matter as conducted to show the generation of isobutanol using genetically modified N. europaea. In some embodiments, the N. europaea is modified using particular genes, which are necessary to produce isobutanol from CO₂. The modified pROBE-NT plasmid was used to screen for the better of two promoters (promoter 1 is the hydroxylamine oxido-reductase promoter (Phao)₅; promoter 2 is the ribulose 1,5 bisphosphate carboxylase/oxygenase large subunit promoter (PrbcL)). As shown in FIG. 14, results indicated that the RubisCO promoter (PrbcL) was more suitable for controlling constitutive expression of the gfp phenotype. A monoclonal cell line that has retained the promoter/reporter construct has been isolated using semi-solid media protocol. This isolation procedure required 3 months of selective growth on semi-solid media. This plasmid and strain has been designated pR-1 and pRNIT-1 respectively.

N. europaea was modified to express 2-keto-acid decarboxylase (Kivd) from Lactococcus lactis with high titer and activity. The kdcA gene (labeled with myc epitope) was inserted into the plasmid under control of the RubisCO promoter. As shown in FIG. 15, initial transformation experiments were performed and trace level production of isobutyraldehyde (˜4.5 mg isobutyraldehyde/L) but no isobutanol) was observed. Confirmation of Kivd expression (in mixed transformed culture) using qualitative-RT-PCR was demonstrated. This plasmid has been designated pRKivd-1.

Still referring to FIG. 15, N. europaea was modified to express alcohol dehydrogenase 2 (Adh2) from Saccharomyces cerevisiae with high titer and activity. The adH2 gene (labeled with myc epitope) was amplified and inserted into the plasmid under control of the RubisCO promoter. Adh2 expression was confirmed using qualitative-RT-PCR. This plasmid has been designated pRADH2-1.

Still referring to FIG. 15, N. europaea was modified to produce isobutanol from CO₂ using a synthetic metabolic pathway. A plasmid was constructed in which the kdcA (labeled with myc epitope) and adH2 (labeled with myc epitope) genes were controlled by the RubisCO promoter separately. This plasmid has been designated pRKA-1. Confirmation of bicistronic expression of both genes was demonstrated.

N. europaea cells were engineered to maximize isobutanol production from CO2. To improve Kivd and ADH2 expression, 3 new plasmids were designed to increase gene transcription:
(1) pRKivd-2—This construct is similar to pRKivd-2 except the five prime untranslated region of the Kivd gene has been modified to include a Shine-Dalgarno sequence unique to Nitrosmonas europaea;
(2) pRADH2-2—This construct is similar to pRADH2-1 except the five prime untranslated region of the ADH2 gene has been modified to include a Shine-Dalgarno sequence unique to Nitrosmonas europaea; and
(3) pRKA-2—This construct is similar to pRKA-1 except the five prime untranslated region of both kdcA and adh2 genes have been modified to include a Shine-Dalgarno sequence unique to Nitrosmonas europaea.

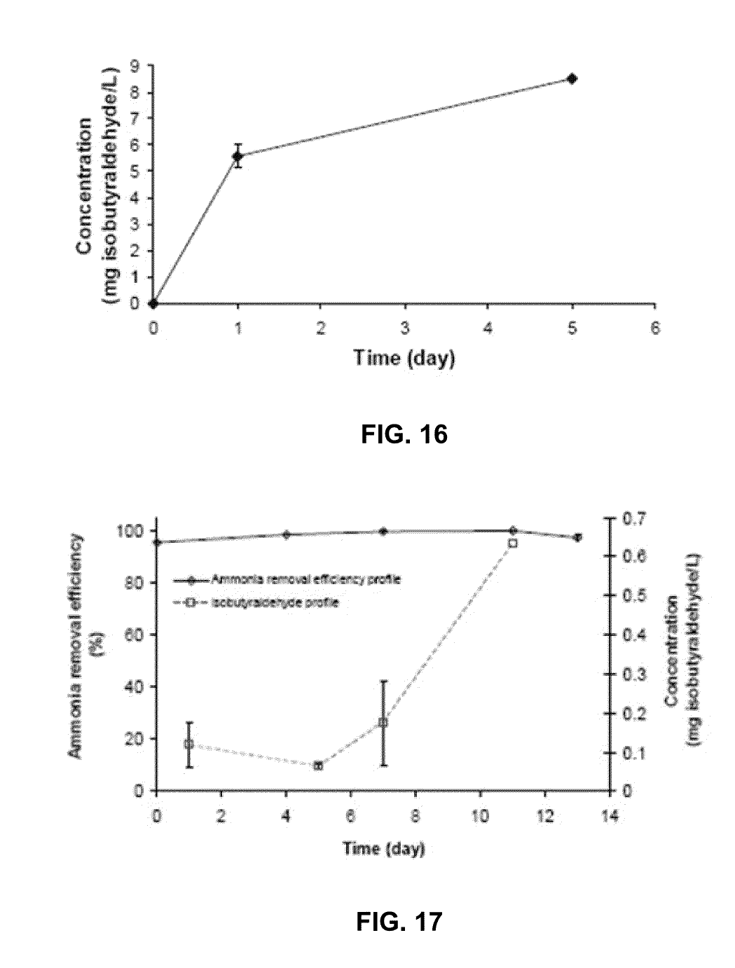

Referring now to FIG. 16, testing confirms the production of isobutyraldehyde (0.038±0.002 isobutyraldehyde/mg N removed) in cultures transformed in the presence of pRKivd-2.

Referring now to FIG. 17, testing of some embodiments according to the disclosed subject matter demonstrated isobutanol production from CO₂ using the genetically modified N. eropaea in a bioreactor combined with the electrochemical production of ammonia. The bioreactor operating with the genetically modified cells under the conditions needed for coupled operation with the electrochemical system was characterized. Continuous flow cultivation of N. europaea cells transformed with pRKivd-1 was performed. Ammonia removal efficiency was been maintained above 97%. Observed biomass yields approached 0.08±0.01 mg biomass as COD/mg N removed. The calculated free energy efficiency averaged 3.9±0.5%. Trace levels of isobutyraldehyde production were observed (Yield=0.0004±0.0005 isobutyraldehyde/mg N removed).

Reverse microbial fuel cells according to the disclosed subject matter utilize carbon dioxide and electrical input to produce infrastructure compatible transportation fuels. The technology uses cultures of AOB, e.g., N. europaea 19718, that are genetically modified to produce isobutanol. The AOB biomass produced from this process is fed to downstream fermentors for additional biofuel production, e.g., n-butanol. Together, the two streams, if combined, have a composition of approximately 90% iso-buantol and 10% n-butanol.

Estimates of e−/e− efficiencies indicate that values significantly greater than one percent are possible. This approach creates a new paradigm for liquid fuel production, and impacts wastewater processes that already utilize N. europaea and other AOB to treat ammonia containing wastewater streams.

Butanol has been noted as being compatible with existing infrastructure. The butanols have liquid fuel energy densities exceeding the 32 MJ/kg. They would likely be used as mixtures in, for example gasoline. Alternatively, the butanol mixtures could be further transformed to other fuels.

Systems and methods according to the disclosed subject matter use only abundant, inexpensive redox mediators. They do not use costly rare earth elements or organic redox shuttles, and thus can be potentially deployed economically at scale. They potentially exceed an overall efficiency greater than one percent and butanol has desirable fuel properties and is compatible with transportation-fuel infrastructure.

The use of ammonia as a mediator in a reverse microbial fuel cell is a significant advancement. Ammonia is extremely attractive for use as a mediator as it is inexpensive. Furthermore, when coupled with a wastewater treatment facility, the oxidation of ammonia to nitrite by the cells is a desirable, revenue generating process. In the absence of a wastewater stream, the nitrite produced by the cells can be reduced back to ammonia using high-throughput flow-through electrodes. The conversion of nitrite to ammonia is at least 80% efficient meaning that low cost electrical energy can be efficiently transferred to the bacterial cells for metabolic processing.

More and more wastewater facilities employ anaerobic digestion for converting the organic fraction of biomass into methane, primarily for pathogen destruction and biosolids treatment. The resulting aqueous stream from anaerobic digestion is disproportionately enriched in organic and ammonia-N (both present in the N(-III) oxidation state). The treatment of this stream is one of the biggest challenges faced by wastewater treatment plants today. Systems and methods according to the disclosed subject matters can utilize ammonia from ammonia-rich streams derived from a wastewater treatment process to not only fix CO₂ using chemolithoautotrophic bacteria, but also converting the fixed CO₂ to a desirable fuel such as isobutanol.

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present invention.

What is claimed is:

1. A method for producing a biofuel using genetically modified ammonia-oxidizing bacteria (AOB), said method comprising:
providing an AOB that have been genetically modified to include a particular metabolic pathway to enable them to generate a particular biofuel;
feeding a first source of ammonia to said AOB;
feeding carbon dioxide to said AOB; and
producing at least said biofuel, nitrite, and an AOB biomass.

2. The method according to claim 1, further comprising:
electrochemically reducing said nitrite to a second source of ammonia; and
feeding said second source of ammonia to said AOB, wherein said second source of ammonia serves as a mediator for transferring electrons to said AOB.

3. The method according to claim 1, wherein said biofuel is isobutanol.

4. The method according to claim 1, wherein said AOB is genetically modified to include at least one of a 2-keto-acid decarboxylase gene and an alcohol dehydrogenase gene.

5. The method according to claim 1, wherein said AOB is substantially Nitrosomonas europaea.

6. The method according to claim 1, further comprising:
fermenting said AOB biomass to produce a mixture including volatile fatty acids; and
fermenting said mixture of volatile fatty acids to produce a second biofuel.

7. The method according to claim 6, wherein said second biofuel is substantially butanol.

8. The method according to claim 6, wherein said mixture of volatile fatty acids includes acetate, propionate, and butyrate.

9. The method according to claim 1, further comprising:
maximizing nitrite production and minimizing nitrate production while producing at least said biofuel, nitrite, and an AOB biomass.

10. The method according to claim 1, wherein said AOB are genetically modified to be able to utilize hydrogen as an electron donor.

11. The method according to claim 10, further comprising:
producing at least one of a long chain alcohol and an alkane.

12. A system for producing biofuels using genetically modified ammonia-oxidizing bacteria, said system comprising:
a bioreactor including AOB that have been genetically modified to include a particular metabolic pathway to enable them to generate a particular biofuel;
a first source of ammonia in fluid communication with said bioreactor;
a source of carbon dioxide in fluid communication with said bioreactor; and
a electrochemical reactor in fluid communication with said bioreactor, said electrochemical reactor configured to electrochemically reduce nitrite produced in said bioreactor to a second source of ammonia.

13. The system according to claim 12, wherein said electrochemical reactor includes a cathode formed substantially from at least one of nickel and glassy carbon.

14. The system according to claim 12, wherein said bioreactor is configured to maximize the production of nitrite and minimize the production of nitrate.

15. The system according to claim 12, wherein said first source of ammonia obtained substantially from an ammonia-rich stream derived from a wastewater treatment process.

16. A method for producing a chemical using genetically modified ammonia-oxidizing bacteria, said method comprising:
providing an AOB that have been genetically modified to include a particular metabolic pathway to enable them to generate a particular chemical;
feeding a first source of ammonia to said AOB;
feeding carbon dioxide to said AOB;
producing at least said chemical, nitrite, and an AOB biomass;
electrochemically reducing said nitrite to a second source of ammonia; and
feeding said second source of ammonia to said AOB.

17. The method according to claim 16, further comprising:
fermenting said AOB biomass to produce a mixture including volatile fatty acids; and
fermenting said mixture of volatile fatty acids to produce a second chemical.

18. The method according to claim 16, wherein said AOB is substantially Nitrosomonas europaea.

19. The method according to claim 16, wherein said AOB are genetically modified to be able to utilize hydrogen as an electron donor.

20. The method according to claim 17, wherein each of said first and second chemicals is one of a commodity chemical, a specialty chemical, a feedstocks such as an acid, an amino acid, a carbohydrate, and a combination thereof.
